(19) United States
(12) Patent Application Publication
(10) Pub. No.: US 2002/0098203 A1
(43) Pub. Date: Jul. 25, 2002

(54) Vaccine composition

(76) Inventors: Nils Ove Gustavsson, Loddekopinge (SE); Monica Jonsson, Bara (SE); Timo Laakso, Campton (GB); Mats Reslow, Lund (SE); Karin Larsson, Torna Hallestad (SE)

Correspondence Address: Benton S. Duffett, Jr., BURNS, DOANE, SWECKER & MATHIS, L.L.P., P.O. Box 1404, Alexandria, VA, 22313-1404, US

(21) Appl. No.: 09/970,794

(22) Filed: Jan. 10, 2002

Related U.S. Application Data
(60) Provisional application No. 60/260,455, filed on Jan. 8, 2001.

(30) Foreign Application Priority Data
Oct. 6, 2000 (SE) 0003615-2

(51) Int. Cl. A61K 39/02; A61K 31/718

(52) U.S. Cl. 424/234.1; 514/60

(57) ABSTRACT

A vaccine composition which comprises an immnunologically active substance embedded in microparticles essentially consisting of starch having an amylopectin content exceeding 85% by weight, of which at least 80% by weight has an average molecular weight within the range of 10-10000 kDa, and without any covalent chemical cross-linking between the starch molecules.

A process for preparing such vaccine composition.

### TECHNICAL FIELD

The present invention lies within the field of galenic formulations for the administration of immunologically active substances, more precisely microparticles for controlled release intended for parenteral administration of immunologically active substances, especially vaccines. More specifically, it relates to a novel production process for such particles containing an immunologically active substance and to novel particles for controlled release and regulation of the immune response obtainable thereby.

### BACKGROUND TO THE INVENTION

One very well established way of preventing disease, or to reduce the consequenses of diseases, is vaccination, which comprises administration of a vaccine, or immunogen or antigen, prior to exposure to the agent causing the disease. A promising use of vaccination which is currently being investigated intensively is the use of therapeutic vaccination to treat diseases which have already broken out.

Many vaccines have to be administered by injection, often repeatedly, since they are either subjected to degradation or are insufficiently absorbed when they are given, for example, orally or nasally or by the rectal route and therefore do not give the desired immune response unless injected into the body.

There is a need for improved immunological adjuvants. Immunological adjuvants are often needed to enhance the desired immune response to obtain sufficient protection from the diseases. It is obviously important for this enhancement that the antigen has retained its native conformation during manufacture of the vaccine preparation and before being released from the injected vaccine preparation.

A vaccine preparation intended for parenteral use has to meet a number of requirements in order to be approved by the regulatory authorities for use on humans. It must therefore be biocompatible and biodegradable and all used substances and their degradation products must be non-toxic, In addition, particulate formulations intended for injection have to be small enough to pass through the injection needle, which preferably means that they should be smaller than 200 μm. The antigen should not be degraded in the preparation to any great extent during production or storage thereof or after administration and should be released in a biologically active form with reproducible kinetics.

One class of polymers which meets the requirements of biocompatibility and biodegradation into harmless end products is the linear polyesters based on lactic acid, glycolic acid and mixtures thereof. These polymers will also hereinafter be referred to as PLGA. PLGA is degraded by ester hydrolysis into lactic acid and glycolic acid and has been shown to possess excellent biocompatibility, The innocuous nature of PLGA can be exemplified, moreover, by the approval by the regulating authorities, including the US Food and Drug Administration, of several parenteral delayed release preparations based on these polymers.

Parenterally administrable delayed release products currently on the market and based on PLGA include Decapeptyl™ (Ibsen Biotech), Prostap SR™ (Lederle), Decapeptyl® Depot (Ferring) and Zoladex® (Zeneca). The drugs in these preparations are all peptides. In other words, they consist of amino acids condensed into a polymer having a relatively low degree of polymerization and they do not have any well-defined three-dimensional structure. This, in turn, usually allows the use of relatively stringent conditions during the production of these products. For example, extrusion and subsequent size-reduction can be utilized, which techniques would probably not be allowed in connection with proteins, since these do not, generally speaking, withstand such stringent conditions.

Consequently, there is also a need for controlled release preparations for proteins. Proteins are similar to peptides in that they also consist of amino acids, but the molecules are larger and the majority of proteins are dependent on a well-defined three-dimensional structure as regards many of their properties, including biological activity and immunogenicity. Their three-dimensional structure can be destroyed relatively easily, for example by high temperatures, surface-induced denaturation and, in many cases, exposure to organic solvents. A very serious drawback connected with the use of PLGA, which is an excellent material per se, for delayed release of proteins is therefore the need to use organic solvents to dissolve the said PLGA, with the attendant risk that the stability of the protein will be compromised and that conformation changes in the protein will lead to an immunological reaction in the patient, which can produce both a loss of therapeutic effect, through the formation of inhibitory antibodies, and toxic side effects. Since it is extremely difficult to determine with certainty whether a complex protein has retained its three-dimensional structure in every respect, it is very important to avoid exposing the protein to conditions which might induce conformation changes.

Despite intense efforts aimed at modifying the PLGA technology in order to avoid this inherent problem of protein instability during the production process, progress within this field has been very slow, the main reason probably being that the three-dimensional structures for the majority of proteins are far too sensitive to withstand the manufacturing conditions used and the chemically acidic environment formed with the degradation of PLGA matrices. The scientific literature contains a large number of descriptions of stability problems in the manufacture of microspheres of PLGA owing to exposure to organic solvents. As an example of the acidic environment which is formed upon the degradation of PLGA matrices, it has recently been shown that the pH value in a PLGA microsphere having a diameter of about 40 μm falls to 1.5, which is fully sufficient to denature, or otherwise damage, many therapeutically usable proteins (Fu et al, Visual Evidence of Acidic Environment Within Degrading Poly(lactic-co-glycolic acid) (PLGA) Microspheres, Pharmaceutical Research, Vol. 17, No. 1, 2000, 100-106). Should the microspheres have a greater diameter, the pH value can be expected to fall further owing to the fact that the acidic degradation products then get more difficult to diffuse away and the autocatalytic reaction is intensified.

The technique which is currently most commonly used to encapsulate water-soluble substances, such as proteins and peptides, is the use of multiple emulsion systems. The drug substance is dissolved in an aqueous or buffer solution and subsequently mixed with an organic solvent, immiscible with water, containing the dissolved polymer. An emulsion is formed which has the aqueous phase as the inner phase. Different types of emulsifiers and vigorous mixing are often used to create this first emulsion. This emulsion is then transferred, under agitation, to another liquid, usually water, containing another polymer, for example polyvinyl alcohol, which produces a water/oil/water triple emulsion. The microspheres are next hardened it some way. The most common way is to utilize an organic solvent having a low boiling point, typically dichloromethane, and to distil off the solvent. If the organic solvent is not fully immiscible with water, a continuous extraction procedure can be used by adding more water to the triple emulsion. A number of variations of this general procedure are also described in the literature. In certain cases, the primary emulsion is mixed with a non-aqueous phase, for example silicone oil. Solid drug materials can also be used instead of dissolved ones.

PLGA microspheres containing proteins are described in WO-Al-9013780, in which the main feature is the use of very low temperatures during the production of the microspheres for the purpose of preserving high biological activity in the proteins. The activity for encapsulated superoxide dismutation is measured, but only on the part which has been released from the particles. This method has been used to produce PLGA microspheres containing human growth hormone in WO-Al-9412158, wherein human growth hormone is dispersed in methylene chloride containing PLGA, the obtained dispersion is sprayed into a container of frozen ethanol beneath a layer of liquid nitrogen in order to freeze the fine droplets and said droplets are allowed to settle in the nitrogen on the ethanol. The ethanol is subsequently thawed and the microspheres start to sink in the ethanol, where the methylene chloride is extracted in the ethanol and the microspheres are hardened. Using this methodology, the protein stability can be better retained than in the majority of other processes for enclosing proteins in PLGA microspheres, and a product has also recently been approved by the regulatory authorities in the USA. However, this still remains to be clearly demonstrated for other proteins and the problem remains of exposing the enclosed biologically active substance to a very low pH during the degradation of the PLGA matrix.

In the aforementioned methods based on encapsulation with PLGA, the active substances are still exposed to an organic solvent and this, generally speaking, is harmful to the stability of a protein. Moreover, the discussed emulsion processes are complicated and probably problematical in any attempt to scale up to an industrial scale. Furthermore, many of the organic solvents which are utilized in many of these processes are associated with environmental problems and their high affinity for the PLGA polymer makes their removal difficult.

A number of attempts to solve the above-described problems caused by exposure of the biologically active substance to a chemically acidic environment during the biodegradation of the microsphere matrix and organic solvents in the manufacturing process have been described. In order to avoid an acidic environment during the degradation, attempts have been made to replace PLGA as the matrix for the microspheres by a polymer which produces chemically neutral degradation products, and in order to avoid exposing the biologically active substance to organic solvents, either it has been attempted to manufacture the microspheres in advance and, only once they have been processed and dried, to load them with the biologically active substance, or attempts have been made to exclude or limit the organic solvent during manufacture of the microspheres.

By way of example, highly branched starch of relatively low molecular weight (maltodextrin, average molecular weight about 5000 Da) has been covalently modified with acryl groups for conversion of this starch into a form which can be solidified into microspheres and the obtained polyacryl starch has been converted into particulate form by radical polymerization in an emulsion with toluene/chloroform (4:1) as the outer phase (Characterization of Polyacryl Starch Microparticles as Carriers for Proteins and Drugs, Artursson et al, J Pharm Sci, 73, 1507-1513, 1984). Proteins were able to be entrapped in these microspheres, but the manufacturing conditions expose the biologically active substance to both organic solvents and high shearing forces in the manufacture of the emulsion. The obtained microspheres are dissolved enzymatically and the pH can be expected to be kept neutral. The obtained microspheres are not suitable for parenteral administrations, especially repeated parenteral administration, for a number of reasons. Most important of all is the incomplete and very slow biodegradability of both the starch matrix (Biodegradable Microspheres IV. Factors Affecting the Distribution and Degradation of Polyacryl Starch Microparticles, Laakso et al, J Pharm Sci 75, 962-967, 1986) and the synthetic polymer chain which cross-links the starch molecules. Moreover, these microspheres are far too small, <2 μm in diameter, to be suitable for injection in the tissues for sustained release, since tissue macrophages can easily phagocytize them. Attempts to raise the degradation rate and the degree of degradation by introducing a potentially biodegradable ester group in order to bond the acryl groups to the highly branched starch failed to produce the intended result and even these polyacryl starch microspheres were biodegraded far too slowly and incompletely over reasonable periods of time (BIODEGRADABLE MICROSPHERES: Some Properties of Polyacryl Starch Microparticles Prepared from Acrylic acid Esterified Starch, Laakso and Sjöholm, 1987 (76), pp. 935-939, J Pharm Sci.)

Microspheres of polyacryl dextran have been manufactured in two-phase aqueous systems (Stenekes et al, The Preparation of Dextran Microspheres in an All-Aqueous System: Effect of the Formulation Parameters on Particle Characteristics, Pharmaceutical Research, Vol. 15, No. 4, 1998, 557-561, and Franssen and Hennink, A novel preparation method for polymeric microparticles without using organic solvents, Int J Pharm 168, 1-7, 1998). with this mode of procedure, the biologically active substance is prevented from being exposed to organic solvents but, for the rest, the microspheres acquire properties equivalent to the properties described for the polyacryl starch microspheres above, which makes them unsuitable for repeated parenteral administrations. Bearing in mind that man does not have specific dextran-degrading enzymes, the degradation rate should be even lower than for polyacryl starch microspheres. The use of dextran is also associated with a certain risk of serious allergic reactions.

Manufacture of starch microspheres with the use of non-chemically-modified starch using an oil as the outer phase has been described (U.S. Pat. No. 4,713,249; Schröder, U., Crystallized carbohydrate spheres for slow release and targeting, Methods Enzymol, 1985 (112), 116-128; Schröder, U., Crystallized carbohydrate spheres as a slow release matrix for biologically active substances, Bio-materials 5:100-104, 1984). The microspheres are solidified in these cases by precipitation in acetone, which leads both to the exposure of the biologically active substance to an organic solvent and to the non-utilization, during the manufacturing process, of the natural tendency of the starch to solidify through physical cross-linking. This leads, in turn, to microspheres having inherent instability, since the starch, after resuspension in water and upon exposure to body fluids, will endeavour to form such cross-links, In order for a water-in-oil emulsion to be obtained, high shear forces are required and the microspheres which are formed are far too small to be suitable for parenteral sustained release.

EP 213303 A2 describes the production of microspheres of, inter alia, chemically unmodified starch in two-phase aqueous systems, utilizing the natural capacity of the starch to solidify through the formation of physical cross-links, and the immobilization of a substance in these microspheres for the purpose of avoiding exposure of the biologically active substance to organic solvents. The described methodology, in combination with the starch quality which is defined, does not give rise to fully biodegradable particles. Neither are the obtained particles suitable for injection, particularly for repeated injections over a longer period, since the described starch quality contains far too high quantities of foreign vegetable protein. In contrast to what is taught by this patent, it has now also surprisingly been found that significantly better yield and higher loading of the biologically active molecule can be obtained if significantly higher concentrations of the polymers are used than is required to form the two-phase aqueous system and that this also leads to advantages in terms of the conditions for obtaining stable, non-aggregated microspheres and their size distribution. The temperature treatments which are described cannot be used for sensitive macromolecules and the same applies to the processing which comprises drying with either ethanol or acetone.

Alternative methods for the manufacture of microspheres in two-phase aqueous systems have been described. In U.S. Pat. No. 5,981,719, microparticles are made by mixing the biologically active macromolecule with a polymer at a pH close to the isoelectric point of the macromolecule and stabilizing the microspheres through the supply of energy, preferably heat. The lowest share of macromolecule, i.e. the biologically active substance, in the preparation is 40%, which for most applications is too high and leads to great uncertainty in the injected quantity of active substance, since the dose of microparticles becomes far too low. Even though the manufacturing method is described as mild and capable of retaining the biological activity of the entrapped biologically active substance, the microparticles are stabilized by heating and, in the examples given, heating is effected to at least 58° C. for 30 min. and, in many cases, to 70-90° C. for an equivalent period, which cannot be expected to be tolerated by sensitive proteins, the biological activity of which is dependent on a three-dimensional structure, and even where the protein has apparently withstood the manufacturing process, there is still a risk of small, but nonetheless not insignificant changes in the conformation of the protein. As the outer phase, a combination of two polymers is always used, generally polyvinyl pyrrolidone and PEG, which complicates the manufacturing process in that both these substances must be washed away from the microspheres in a reproducible and reliable manner. The formed microparticles are too small (in the examples, values below 0.1 μm in diameter are quoted) to be suitable for parenteral sustained release after, for example, subcutaneous injection, since macrophages, which are cells which specialize in phagocytizing particles and which are present in the tissues, are easily capable of phagocytizing microspheres up to 5-10, possibly 20 μm, and the phagocytized particles are localized intracellularly in the lysosomes, where both the particles and the biologically active substance are degraded, whereupon the therapeutic effect is lost. The very small particle size also makes the processing of the microspheres more complicated, since desirable methods, such as filtration, cannot be used. The equivalent applies to U.S. Pat. No. 5,849,884.

U.S. Pat. No. 5,578,709 and EP 0 688 429 B1 describe the use of two-phase aqueous systems for the manufacture of macromolecular microparticle solutions and chemical or thermal cross-linking of the dehydrated macromolecules to form microparticles. It is entirely undesirable to chemically cross-link the biologically active macromolecule, either with itself or with the microparticle matrix, since chemical modifications of this kind have a number of serious drawbacks, such as reduction of the bioactivity of a sensitive protein and risk of induction of an immune response to the new antigenic determinants of the protein, giving rise to the need for extensive toxicological studies to investigate the safety of the product. Microparticles which are made through chemical cross-linking with glutaraldehyde are previously known and are considered generally unsuitable for repeated administrations parenterally to humans. The microparticles which are described in U.S. Pat. No. 5,578,709 suffer in general terms from the same drawbacks as are described for U.S. Pat. No. 5,981,719, with unsuitable manufacturing conditions for sensitive proteins, either through their exposure to chemical modification or to harmful temperatures, and a microparticle size distribution which is too narrow for parenteral, sustained release and which complicates post-manufacture processing of the microspheres.

WO 97/14408 describes the use of air-suspension technology for producing microparticles for sustained release after parenteral administration, without the biologically active substance being exposed to organic solvents. However, the publication provides no guidance towards the process according to the invention or towards the novel microparticles which can thereby be obtained.

In U.S. Pat. No. 5,470,582, a microsphere consisting of PLGA and containing a macromolecule is produced by a two-stage process, in which the microsphere as such is first manufactured using organic solvents and then loaded with the macromolecule at a later stage in which the organic solvent has already been removed. This procedure leads to far too low a content of the biologically active substance, generally 1-2%, and to a very large fraction being released immediately after injection, which very often is entirely unsuitable. This far too rapid initial release is already very high given a 1% load and becomes even more pronounced when the active substance content in the microspheres is higher, Upon the degradation of the PLGA matrix, the pH falls to levels which are generally not acceptable for sensitive macromolecules.

That starch is, in theory, a very suitable, perhaps even ideal, matrix material for microparticles has been known for a long time, since starch does not need to be dissolved in organic solvents and has a natural tendency to solidify and since there are enzymes within the body which can break down the starch into endogenic and neutral substances, ultimately glucose, and since starch, presumably owing to the similarity with endogenic glycogen, has been shown to be non-immunogenic. Despite intense efforts, starch having properties which enable manufacture of microparticles suitable for parenteral use and conditions which enable manufacture of fully biodegradable microparticles under mild conditions, which allow sensitive, immunologically active substances, such as proteins, to become entrapped, has not been previously described.

Starch granules naturally contain impurities, such as starch proteins, which makes them unsuitable for injection parenterally. In the event of unintentional depositing of insufficiently purified starch, such as can occur in operations where many types of operating gloves are powdered with stabilized starch granules, very serious secondary effects can arise. Neither are starch granules intrinsically suitable for repeated parenteral administrations, for the reason that they are not fully biodegradable within acceptable time spans.

Starch microspheres made of acid-hydrolyzed and purified starch have been used for parenteral administration to humans. The microspheres were made by chemical cross-linking with epichlorohydrin under strongly alkaline conditions. The chemical modification which was then acquired by the starch leads to reduced biodegradability, so that the microspheres can be fully dissolved by endogenic enzymes, such as α-amylase, but not converted fully into glucose as the end product. Neither the manufacturing method nor the obtained microspheres are suitable for the immobilization of sensitive proteins, and as is evident from the control experiments, nor is acid-hydrolyzed starch suitable for producing either fully biodegradable starch microspheres or starch microspheres containing a high load of a biologically active substance, such as a protein.

Hydroxyethyl starch (HES) is administered parenterally to humans in high doses as a plasma substitute. HES is produced by starch granules from starch consisting broadly exclusively of highly branched amylopectin, so-called “waxy maize”, being acid-hydrolyzed in order to reduce the molecular weight distribution and being subsequently hydroxyethylated under alkaline conditions and acid-hydrolyzed once more to achieve an average molecular weight of around 200,000 Da. After this, filtration, extraction with acetone and spray-drying are carried out. The purpose of the hydroxyethylation is to prolong the duration of the effect, since non-modified amylopectin is very rapidly degraded by α-amylase and its residence time in the circulation is ca. 10 minutes. HES is not suitable for the production of fully biodegradable microspheres containing a biologically active substance, since the chemical modification leads to a considerable fall in the speed and completeness of the biodegradation and results in the elimination of the natural tendency of the starch to solidify through the formation of non-covalent cross-linkings. Moreover, highly concentrated solutions of HES become far too viscous to be usable for the production of microparticles. The use of HES in these high doses shows that parenterally usable starch can be manufactured, even though HES is not usable for the manufacture of microspheres without chemical cross-linking or precipitation with organic solvents.

WO 99/00425 describes the use of heat-resistant proteolytic enzymes with wide pH-optimum to purge starch granules of surface-associated proteins. The obtained granules are not suitable for parenteral administration, since they still contain the starch proteins which are present within the granules and there is a risk that residues of the added proteolytic enzymes will be left in the granules. Neither are the granules suitable for the manufacture of parenterally administrable starch microspheres in two-phase aqueous systems, since they have the wrong molecular weight distribution to be able to be used in high enough concentration, even after being dissolved, and, where microspheres can be obtained, they are probably not fully biodegradable.

The use of shearing to modify the molecular weight distribution of starch, for the purpose of producing better starch for the production of tablets, is described in U.S. Pat. No. 5,455,342 and WO 93/21008. The starch which is obtained is not suitable for parenteral administration owing to the high content of starch proteins, which might be present in denatured form after the shearing, and neither is the obtained starch suitable for producing biodegradable starch microspheres for parenteral administration or for use in two-phase aqueous systems for the production of such starch microspheres. Shearing has also been used to manufacture hydroxyethylstarch, as is disclosed in WO 96/10042. However, for similar reasons such hydroxyethylstarch is not either suitable for parenteral administration or for the production of microspheres as referred to above.

In several of the documents describing controlled release preparations of drugs evaluated above vaccines or antigens are mentioned as a potential class of substances to use, for example in U.S. Pat. Nos. 5,849,884, 5,981,719, WO 99/20253, EP 688 429, U.S. Pat. Nos. 4,713,249, 5,470,582. For each of these the evaluation remains the same except for that the need for a large particle size, so as to avoid macrophage phagocytosis, which is critical for controlled release of, for example, therapeutic protein drugs, may in some cases not be so crucial, or a smaller particle size may even be desirable, as phagocytosis of antigens is one starting point for obtaining an immune response for many antigens.

In U.S. Pat. No. 5,753,234 a vaccine formulation comprising a core consisting of hydroxypropyl cellose or sodium carboxymethyl cellulose or gelatin, and containing an antigen, and a biodegradable coating is disclosed. The coating is obtained by dispersing the core particles in an organic solvent containing the biodegradable polymer, for example PLGA, and applied by spray drying. Neither hydroxypropyl cellose nor sodium carboxymethyl cellulose are biodegradable and gelatin is unsuitable due to the risk of immune responses. A serious drawback with the preparation process is the exposure of the core containing the antigen to organic solvents. Although it has been demonstrated that the core is able to protect HbsAg from organic solvents like ethyl acetate and acetonitrile, this has not been demonstrated for other antigens and it is very undesirable to expose the antigen to an organic solvent at all since this may be harmful to the antigen and may result in residual solvent in the formulation which may adversely affect the stability of the formulation in general, and the antigen in particular. It is also not desirable to control the release kinetics by the thickness of the coating as this limits the release profiles that are achievable and limits the ratio between the core and the coating that can be used.

A process for the production of parenterally administrable starch preparations having the following features would therefore be extremely desirable:

a process which makes it possible to entrap sensitive, immunologically active substances, for example antigens, in microparticles to obtain a vaccine preparation, with retention of their immunological activity;

a process by means of which immunologically active substances can be entrapped under conditions which do not expose them to organic solvents, high temperatures or high shear forces and which allows them to retain their immunological activity;

a process which permits high loading of a parenterally administrable preparation with even sensitive, immunologically active substances;

a process by means of which a substantially fully biodegradable and biocompatible preparation can be produced, which is suitable for injecting parenterally and upon whose degradation chemically neutral endogenic substances are formed;

a process by means of which a parenterally injectable preparation having a size exceeding 20 μm and, preferably exceeding 30 μm, can be produced for the purpose of avoiding phagocytosis of tissue macrophages and which simplifies processing of the same during manufacture;

a process for the production of microparticles containing an immunologically active substance, which microparticles can be used as intermediate product in the production of a preparation for controlled, sustained or delayed release and which permit rigorous quality control of the chemical stability and immunological activity of the entrapped immunological substance;

a process which utilizes a parenterally acceptable starch which is suitable for the production of substantially fully biodegradable starch microparticles;

a substantially fully biodegradable and biocompatible microparticulate preparation which is suitable for injecting parenterally and upon whose degradation chemically neutral endogenic substances are formed;

a microparticulate preparation containing an immunologically active substance and having a particle size distribution which is suitable for coating by means of air suspension technology and having sufficient mechanical strength for this purpose.

Objects such as these and other objects are achieved by means of the invention defined below.

### DESCRIPTION OF THE INVENTION

According to one aspect of the present invention, it relates to a process for production of microparticles. More specifically it relates to production of microparticles which contain at least one immunologically active substance and which are intended for parenteral administration of the said substance to a mammal, especially a human, as a vaccine preparation. The said parenteral administration primarily means that the microparticles are intended for injection.

The immunologically active substance, which may also be named immunogen, antigen or immunizing agent, is a substance having the ability to induce a desired immune response when administered alone, or in combination with at least one suitable adjuvant. This immune response can be humorally and/or cellularly mediated in a mammal, preferably man. In general, the substance referred to, does not possess any biological or pharmacological activity immediately, or a relatively short period of time after administration, at least the first time it is administered, but its desirable action is mediated by stimulating the recipient's immune system to form an immune response. This process normally takes at least one week and often substantially longer, to confer a sufficient protection. In many cases it is necessary to repeat the administration with suitable time intervals to biuld up a sufficient protection.

Since the microparticles are primarily intended for injection, it is a question especially of manufacturing particles with an average diameter within the range of 1-200 μm, generally 20-100 μm and in particular 20-80 μm, when the immunogen is intended for controlled release and <10 μm when the immunogen is intended for phagocytosis.

The expression “microparticles” is used in connection with the invention as a general designation for particles of a certain size known in the art. One type of microparticles is that of microspheres which have in the main a spherical shape, although the term microparticle may generally include deviations from such an ideal spherical shape. The term microcapsule known in the art is also covered by the expression microparticle in accordance with the known art.

The process according to the present invention more specifically comprises:

a) preparing of an aqueous starch solution containing starch, which has an amylopectin content in excess of 85 percent by weight, in which the molecular weight of said amylopectin has been reduced such that at least 80 percent by weight of the material lies within the range of 10-10000 kDa, the starch concentration of the solution being at least 20 percent by weight,

b) combining the immunologically active substance with the starch solution under conditions such that a composition is formed in the form of a solution, emulsion or suspension of said substance in the starch solution,

c) mixing the composition obtained in step b) with an aqueous solution of a polymer having the ability to form a two-phase aqueous system, so that an emulsion of starch droplets is formed which contain the immunologically active substance as an inner phase in an outer phase of said polymer solution,

d) causing or allowing the starch droplets obtained in step c) to gel into starch particles through the natural propensity of the starch to solidify,

e) drying the starch particles, and

f) optionally applying a release-controlling shell of a biocompatible and biodegradable polymer, preferably by an air suspension method, to the dried starch particles.

An important aspect of this process is, in other words, the use of a certain type of starch as microparticle matrix. One starch that is especially suitable, and a process for the production thereof, are described in the Swedish patent application No. 0003616-0. In this case the molecular weight reduction is accomplished by shearing. Another useful starch is disclosed in a PCT application copending to the present application and entitled STARCH.

In last-mentioned case the molecular weight reduction is accomplished by acid hydrolysis.

Details about the starch may in other words be obtained from said patent applications, the contents of which are thus in this respect introduced into the present text by way of reference.

Some further important features of such a starch will, however, be described below. In order that fully biodegradable microparticles with high active substance yield shall be formed in a two-phase aqueous system and in order that the obtained starch microparticles shall have the properties to be described below, the starch must generally predominantly consist of highly branched starch, which, in the natural state in the starch granule, is referred to as amylopectin. It should also have a molecular weight distribution which makes it possible to achieve desired concentrations and gelation rates.

It may be added, in this context, that the term “biodegradable” means that the microparticles, after parenteral administration, are dissolved in the body to form endogenic substances, ultimately, for example, glucose. The biodegradability can be determined or examined through incubation with a suitable enzyme, for example alpha-amylase, in vitro. It is in this case appropriate to add the enzyme a number of times during the incubation period, so as thereby to ensure that there is active enzyme permanently present in the incubation mixture. The biodegradability can also be examined through parenteral injection of the microparticles, for example subcutaneously or intramuscularly, and histological examination of the tissue as a function of time.

Biodegradable starch microparticles disappear normally from the tissue within a few weeks and generally within one week. In those cases in which the starch microparticles are coated with a release-controlling shell, for example coated, it is generally this shell which determines the biodegradability rate, which then, in turn, determines when alpha-amylase becomes available to the starch matrix.

The biocompatibility can also be examined through parenteral administration of the microparticles, for example subcutaneously or intramuscularly, and histological evaluation of the tissue, it being important to bear in mind that the immunologically active substance, which often is a protein, has in itself the capacity to induce, for example, an immunodefence if administered in another species.

The starch must further have a purity which is acceptable for the manufacture of a parenterally administrable preparation. It must also be able to form sufficiently stable solutions in sufficiently high concentration to enable the immunologically active substance to be mixed in under conditions allowing the retention of the immunological activity of the substance, at the same time as it must spontaneously be able to be solidified in a controlled manner in order to achieve stable, yet at the same time biodegradable, microparticles. High concentration of the starch is also important to prevent the immunologically active substance from being distributed out to an unacceptable extent to the outer phase or to the interface between the inner and the outer phases.

A number of preferred embodiments with regard to the character of the starch are as follows.

The starch preferably has a purity of less than 50 μg, more preferably at most 20 μg, even more preferably at most 10 μg, and most preferably at most 5 μg, amino acid nitrogen per g dry weight of starch.

The molecular weight of the abovementioned amylopectin is preferably reduced, such that at least 80% by weight of the material lies within the range of 100-4000 kDa, more preferably 200-1000 kDa, and most preferably 300-600 kDa.

In addition, the starch preferably has an amylopectin content with the reduced molecular weight in question exceeding 95% by weight, more preferably exceeding 98% by weight. It can also, of course, consist of 100% by weight of such amylopectin.

According to another preferred embodiment, the starch is of such a type that it can be dissolved in water in a concentration exceeding 25% by weight. This means, in general, a capacity to dissolve in water according to a technique which is known per se, i.e. usually dissolution at elevated temperature, for example up to approximately 80° C.

According to a further preferred embodiment, the starch is substantially lacking in covalently bonded extra chemical groups of the type which are found in hydroxyethyl starch. By this is meant, in general, that the starch essentially only contains groups of the type which are found in natural starch and have not been in any way modified, such as in hydroxyethyl starch, for example.

Another preferred embodiment involves the starch having an endotoxin content of less than 25 EU/g.

A further preferred embodiment involves the starch containing less than 100 microorganisms per gram, often even less than 10 microorganisms per gram.

The starch can further be defined as being substantially purified from surface-localized proteins, lipids and endotoxins by means of washing with aqueous alkali solution, reduced in molecular weight by means of shearing, and purified from internal proteins, for example by means of electrophoresis or ion exchange chromatography, preferably anion exchange chromatography.

As far as the purity in all these contexts is concerned, it is in general the case that expressions of the type “essentially” or “substantially” generally mean to a minimum of 90%, for example 95%, 99% or 99.9%.

That amylopectin constitutes the main component part in the starch used means in general terms that its share is 90-100% by weight, calculated on the basis of dry weight of starch.

In certain cases, it can here be favourable to use a lesser share, for example 2-15% by weight, of short-chain amylose to modify the gelation rate in step d). The average molecular weight of the said amylose lies preferably within the range of 2.5-70 kDa, especially 5-45 kDa. Other details regarding short-chain amylose can be obtained from U.S. Pat. No. 3,881,991.

In the formation of the starch solution in step a), heating according to a technique which is known per se is in general used to dissolve the starch. An especially preferred embodiment simultaneously involves the starch being dissolved under autoclaving, it also preferably being sterilized. This autoclaving is realized in aqueous solutions, for example water for injection or suitable buffer.

If the immunologically active substance is a sensitive protein or another temperature-sensitive substance, the starch solution must cool to an appropriate temperature before being combined with the substance in question. What temperature is appropriate is determined firstly by the thermal stability of the immunologically active substance, but in purely general terms a temperature of less than ca. 60° C., preferably less than 55° C., is appropriate.

According to a preferred embodiment, the active substance is therefore combined with the starch solution at a temperature of at most 60° C., more preferably at most 55° C., and most preferably within the range of 20-45° C., especially 30-37° C.

For the mixing operation in step b), furthermore, a weight ratio of starch:immunologically active substance within the range of 3:1 to 10000:1, or preferably 3:1 to 100:1, is expediently used.

It is also the case for the mixing operation that the active substance is mixed with the starch solution before a two-phase aqueous system is formed in step c). The active substance can be in dissolved form, for example in a buffer solution, or in solid, amorphous or crystalline form, and at a suitable temperature, which is generally between room temperature (20° C.) and 45° C., preferably at most 37° C. It is possible to add the starch solution to the immunologically active substance, or vice versa. Since the immunologically active substances suitable for use in this system, for example proteins, are generally macromolecules, it is possible, when mixing a solution of a dissolved macromolecule with starch, for an emulsion to form, in which the macromolecule generally represents the inner phase, or a precipitate. This is entirely acceptable, provided that the immunologically active substance retains or does not appreciably lose its immunological activity. A homogeneous solution, emulsion or suspension is then created by agitation, which can be carried out using a suitable technique Such a technique is well known within the field, examples which might be quoted being magnetic agitation, propeller agitation or the use of one or more static mixers. An especially preferred embodiment of the invention is represented in this case by the use of at least one static mixer.

In the production of the starch microparticles according to the present invention, the concentration of starch in the solution which is to be converted to solid form and in which the immunologically active substance is to be incorporated should be at least 20% by weight to enable the formation of starch microparticles having good properties. Exactly that starch concentration which works best in each individual case can be titrated out in a simple manner for each individual immunologically active substance, where the load in the microparticles is that which is required in the individual case. In this context, it should be noted that the immunologically active substance to be incorporated in the microparticles can affect the two-phase system and the gelation properties of the starch, which also means that customary preparatory trials are conducted for the purpose of determining the optimal conditions in the individual case, Trials generally show that the starch concentration should advantageously be at least 30% by weight and in certain specific cases at least 40% by weight. As the highest limit, 50% by weight is usually applicable, especially at most 45% by weight. It is not normally possible to obtain these high starch concentrations without the use of molecular-weight-reduced, highly branched starch.

Regarding the polymer used in step c) for the purpose of forming a two-phase aqueous system, information is published, within precisely this technical field, on a large number of polymers with the capacity to form two-phase systems with starch as the inner phase. All such polymers must be considered to lie within the scope of the present invention. An especially suitable polymer in this context, however, is polyethylene glycol. This polyethylene glycol preferably has an average molecular weight of 5-35 kDa, more preferably 15-25 kDa and especially about 20 kDa.

The polymer is dissolved in suitable concentration in water or aqueous solution, which expression also includes buffer solution, and is temperature-adjusted to a suitable temperature. This temperature lies preferably within the range of 4-50° C., more preferably 10-40° C. and most preferably 10-37° C. The concentration of the polymer in the aqueous solution is at least 20% by weight and preferably at least 30% by weight, and more expediently at most 45% by weight. An especially preferred range is 30-40% by weight.

The mixing operation in step c) can be executed in many different ways, for example through the use of propeller agitation or at least one static mixer. The mixing is normally carried out within the temperature range of 4-50° C., preferably 20-40° C., often about 37° C. In a batch process, the starch solution can be added to the polymer solution or vice versa. Where static mixers or blenders are utilized, the operation is expediently executed by the two solutions being pumped in two separate pipelines into a common pipeline containing the blenders.

The emulsion can be formed using low shearing forces, since there is no high surface tension present between the phases in water/water emulsions, in contrast to oil/water or water/oil emulsions, and in this case it is primarily the viscosity of the starch solution which has to be overcome for the droplets to achieve a certain size distribution. In most cases, magnetic or propeller agitation is sufficient. On a larger scale, for example when the quantity of microparticles to be produced exceeds 50 g, it is expedient to use so-called baffles to obtain even more effective agitation in the container which is used. An alternative way of forming the water/water emulsion is to use at least one static mixer, the starch solution expediently being pumped at regulated speed in a pipe in which the static mixers have been placed. The pumping can be effected with any type of suitable pump, provided that it gives an even flow rate under these conditions, does not expose the mixture to unnecessarily high shear forces and is acceptable for the manufacture of parenteral preparations in terms of purity and non-leakage of unwanted substances. In those cases, too, in which static mixers are used to create the emulsion, it is generally advantageous to have the solidification into microparticles take place in a vessel with suitable agitation.

A preferred embodiment of the process according to the invention means that in step c) the polymer solution is added to the composition in at least two stages, in which an admixture is effected after the emulsion has been created or has begun to be created.

It is also within the scope of the present invention, of course, to add the polymer solutions in many stages and to change, for example, the average molecular weight and/or concentration of the polymer used, for example in order to increase the starch concentration in the inner phase where this is desirable.

The mixing operation in step c) is also expediently executed under such conditions that the starch droplets formed have the size required for the microparticles, i.e. preferably a mean diameter, in the dry state, within the range of 1-200 μm, generally 20-100 μm, and in particular 20-80 μm, when the immunogen is intended for controlled release and <10 m when the immunogen is intended for phagocytosis.

In the production of the microparticles according to the present invention it is essential that the solidification occurs through the natural tendency or capacity of the starch to gel and not, for example, through precipitation with organic solvents, such as acetone. The latter procedure may lead to the immunologically active substance being exposed to organic solvent, which in many cases is unacceptable, and to an absence of the natural formation of the physical cross-linkages that are required in order to obtain stable microparticles in a controlled manner.

In connection with the solidification of the microparticles, it is important that this should take place under conditions which are mild for the incorporated immunologically active substance(s). In other words, it is primarily a question of using a temperature which is not harmful to the current substance. In this context, it has surprisingly been shown that the criteria for this and for the formation of stable microparticles with suitable size distribution can more easily be met if, during the solidification, more than one temperature or temperature level is used. It is especially advantageous if the solidification process in the two-phase system is initiated at a lower temperature than the temperature which is used in the end phase of the solidification. A preferred embodiment means that the solidification is initiated within the range of 1-20° C., preferably 1-10° C., especially around 4° C., and is concluded within the range of 20-55° C., preferably 25-40° C., especially around 37° C.

Confirmation that the chosen conditions are correct or appropriate can be obtained by establishing that the starch microparticles have a desired size distribution, are stable during the subsequent washing and drying operations and are dissolved substantially by fully enzymatic means in vitro and/or that the incorporated substance has been encapsulated effectively and has retained activity. The last-mentioned is usually examined using chromatographic methods or using other methods established within the art, in vitro or in vivo, after the microparticles have been enzymatically dissolved under mild conditions, and is an important element in ensuring a robust and reliable manufacturing process for sensitive, immunologically active substances. It is a great advantage for the microparticles to be able to be fully dissolved under mild conditions, since this minimizes the risks of preparation-induced artifacts, which are usually found when, for example, organic solvents are required to dissolve the microparticles, which is the case, for example, when these consist of a PLGA matrix.

The formed microparticles are preferably washed in a suitable manner in order to remove the outer phase and any surplus active substance. Such washing is expediently effected by filtration, which is made possible by the good mechanical stability and suitable size distribution of the microparticles. Washing by means of centrifugation, removal of the supernatant and resuspension in the washing medium may often also be appropriate. In each washing process, one or more suitable washing media are used, which generally are buffer-containing aqueous solutions. In this connection, sieving can also be used, if required, in order to adjust the size distribution of the microparticles, for example to eliminate the content of too small microparticles and to ensure that no microparticles above a certain size are present in the finished product.

The microparticles can be dried in any way appropriate, for example by spray-drying, freeze-drying or vacuum-drying. Which drying method is chosen in the individual case often depends on what is most appropriate for the retention of the immunoactivity for the encapsulated immunologically active substance. Process considerations also enter into the picture, such as capacity and purity aspects. Freeze-drying is often the preferred drying method, since, correctly designed, it is especially mild with respect to the entrapped immunologically active substance. That the incorporated immunologically active substance has retained its bioactivity can be established by means of analysis appropriate to the microparticle after the substance has been enzymatically dissolved under mild conditions. Suitable enzymes for use in connection with starch are alpha-amylase and amyloglucosidase, singly or in combination, it being important to establish, where appropriate, that they are free from possible proteases, which can degrade proteins. The presence of proteases can be detected with methods known within the field and, for example, by mixing the immunologically active substance in control trials and determining its integrity in the usual manner after incubation with the intended enzyme mixture under the conditions which will afterwards be used to dissolve the microparticles. Where the preparation is found to contain protease contamination, it can be replaced by a preparation which offers higher purity or is purified from proteases. This can be done using techniques known within the field, for example by chromatography with α₂.macroglobulin bonded to suitable chromatographic material.

In order to modify the release properties for the microparticles, a release-controlling shell, or coating, made from a biocompatible and biodegradable polymer might also be applied. Examples of suitable polymers in this context are found in the prior art, and polymers of lactic acid and glycolic acid (PLGA) can especially be mentioned. Polymers able to provide an enteric coating can also be used and examples of suitable polymers can be found in the prior art. The shell in question is preferably applied using air suspension technology. An especially suitable technique of this kind is described in WO97/14408 and details in this regard can thus be obtained from this publication, the content of which is included in the text by reference. The starch microparticles which are obtained by means of the process according to the present invention are extremely well suited to coating or coating by means of the said air suspension technology, and the coated microparticles obtained are especially well suited to parenteral administration.

When the produced microparticles are used, either they are coated with a release-controlling outer shell or not, and the dry microparticles are suspended in a suitable medium, specifically to permit injection. Such media and processes in these regards are well known within the field and will not need here to be described in further detail. The actual injection can be given through a suitable needle or with a needle-free injector. It is also possible to inject the microparticles using a dry powder injector, without prior resuspension in an injection medium.

Apart from the advantages which have been discussed above, the process according to the invention has the advantage that the yield of the immunologically active substance is generally high, that it is possible to obtain a very high active substance content in the microparticles whilst retaining the immunoactivity of the substance, and that endogenic and neutral degradation products are formed upon degradation of the microparticles, by which means the active substance, for example, can be prevented from being exposed to an excessively low pH value. Moreover, the process itself is especially well suited to rigorous quality control.

It is also a great advantage that the size of the microparticles can be adjusted to meet the requirements for each particular antigen in that large microparticles can be made for parenteral, controlled (for example delayed or sustained) release of the vaccine component, thus combining the properties of being too large to be phagocytized by macrophages and small enough to be injectable through small needles, for example 23G-25G, or, if desired, small particle can be made if it is desired that the vaccine component is phagocytosed by macrophages. Obviously a combination of large and small particles can be used if it is desirable to use both controlled release and macrophage phagocytosis to induce an immune response.

A further advantage is the possibility of designing the release kinetics of the vaccine component so as to suit each unique vaccine component, primarily by adjusting the composition of the coating. One type of release that can be achieved is a continuous, essentially or almost, linear release, with, if necessary, only a small fraction released initially during the release phase. Another type of release that can be achieved is a biphasic release, in which one fraction of the entrapped vaccine component is released initially during the release phase, followed by a phase of no, or very low, release, and then a second phase where the remaining vaccine component is released, often during a comparatively short time period.

The process according to the invention is especially interesting in connection with proteins, peptides, polypeptides, polynucleotides and polysaccharides or, in general, other antigens or immunologically active substances which are sensitive to or unstable in, for example, organic solvents. These vaccine components can be either water soluble or consist of particles, whole killed organisms, etc, which are not water soluble. Recombinantly produced proteins are a very interesting group of immunologically active substances, Generally speaking, however, the invention is not limited to the presence of such substances, since the inventive concept is applicable to any immunologically active substance which can be used for parenteral administration. Apart from in connection with sensitivity or instability problems, the invention can thus also be of special interest in such cases where it would otherwise be difficult to remove solvent or where toxicological or other environmental problems might arise.

The following classes of vaccines are particularly interesting to prepare in accordance with the invention: NMDA glutamate receptor vaccines, whole cell vaccines, tumour-antigen vaccines, peptide vaccines, allergoid vaccines, anti-iodiotype vaccines, dendritic cell-based vaccines, subunit and recombinant subunit vaccines, DNA vaccines, live viral vector vaccines, live bacterial vaccines and self antigen vaccines.

Vaccine components, i.e. immunologically active substances, that can be encapsulated by the process claimed include allergens such as cat dander, animal dander, flower pollens, weed pollens, tree pollens like birch pollen, house dust mite, grass pollen, and the like, mould allergy entigens; antigens of such bacterial organisms as Streptococcus pneumoniae, Haemophilus influenzae, staphylococcus aureus, Streptococcus porygenes, Corynebacterium diphtheriae, Listeria monocytogenea, Bacillus anthracis, Clostridium tetani, Clostridium botulinum, Clostridium perfringens, Neisseria meningitides, Neisseria gonnorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema palladium, Leptspirosis interrogans, Borrelia burgdorferi, Campylobacter jejuni, and the like; antigens of such viruses as smallpox, influenza A and B, respiratory syncytial, parainfluenza, measles, HIV, varicella-zoster, herpes simplex 1 and 2, cytomeglavirus, Epstein-Barr, rotavirus, rhinovirus, adenovirus, papillomavirus, poliovirus, mumps, rabies, rubella, coxsackieviruses, equine encephalitis, Japanese encephalitis, yellow fever, Rift Valley fever, lymphocytic choriomeningitis, hepatitis B, and the like; antigens of such fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In the present invention any adjuvant that can either be encapsulated in the microparticle preparation or added to the injection vehicle can be used. A non limiting list can be found in “Vaccine Design: the subunit and adjuvant approach”, ed. Michael F. Powell and Mark J. Newman (Pharmaceutical biotechnology; v6), Plenum Press, New York 1995, ISBN- 0-306-44867-X. However, some preferable adjuvants are such adjuvants which are derived from mineral salts, especially aluminium salts, saponins, lipid-A or analogues or derivates thereof, immunostilmulatory oligonucleotides, cytokines and low molecular weight thiols, for example cysteamine.

Many diseases are suitable for therapeutic vaccination and the following is a non-limiting list of diseases to be treated by the vaccine composition according to the present invention; insulin-dependent diabetes mellitus (Type 1), autoimmune diseases, allergy and asthma, cancer, cardiovascular disease and many other chronic disease, for example infectious diseases, rheumatoid arthritis and neurodegenerative disorders.

The vaccine composition can also be prepared by a process which comprises

a) preparing an aqueous solution of the immunologically active substance,

b) combining the immunologically active substance with starch microspheres under such conditions that the antigen becomes associated with the microspheres,

c) evaporation or other drying.

According to another aspect of the invention, it also relates to novel microparticles of the type which are obtainable by means of the process according to the invention. The novel microparticles according to the invention are not limited, however, to those which can be produced by means of the said process, but comprise all microparticles of the type in question irrespective of the production methods.

More specifically, these are microparticles suitable for parenteral administration, preferably by way of injection, to a mammal, especially a human, and containing an immunologically active substance embedded therein, which microparticles consist substantially of starch that has an amylopectin content in excess of 85 percent by weight, of which at least 80 percent by weight has an average molecular weight in the range 10-10000 kDa.

The starch preferably has an amino acid content of less than 50 μg per dry weight of starch and there is no covalent chemical cross-linking between the starch molecules.

The starch on which the microparticles in question are based is preferably one of the types of starch defined above in connection with the process.

According to a preferred embodiment of the microparticles according to the invention, the immunoactivity of the immunologically active substance in these is at least 80%, preferably at least 90% of the immunogenicity that the substance exhibited before it was incorporated into the starch. The said immunogenicity is most preferably largely retained or preserved in the microparticles.

Yet another preferred embodiment of the invention is represented by microparticles which are biodegradable in vitro in the presence of α-amylase and/or amyloglucosidase.

Another embodiment is represented by those that are biodegradable and are eliminated from tissue after subcutaneous or intramuscular administration.

An especially preferred embodiment of the microparticles is represented by particles which have a release-controlling shell of at least one film-forming, biocompatible and biodegradable polymer able to provide sustained release of the immunologically active agent, or in other words generally a release such that upon suspension in an aqueous medium at physiological conditions over 50% of the associated antigen is released within 3 hours.

The said polymer is preferably a homopolymer or copolymer made from α-hydroxy acids, the said α-hydroxy acid preferably being lactic acid and/or glycolic acid. Another variant is cyclic dimer of an α-hydroxy acid which is preferably selected from the group consisting of glycolides and lactides.

Such polymers or dimers (of the PLGA type, for example) are precisely described in the prior art, and further details of these may therefore be obtained therefrom.

Another embodiment is represented by microparticles in which, in addition to said polymer, the shell contains at least one release regulating substance. Such a substance is preferably water soluble or sparingly water soluble. It is preferably selected from lactic acid, oligomers containing lactic acid and glycolic acid.

It may also advantageously be selected from substances comprising polyethylene glycol (PEG) and block copolymers comprising PEG as one of the blocks.

Another interesting embodiment is represented by microparticles which have an outer layer of at least one water soluble substance having the ability to prevent aggregation of the microparticles.

A further preferred embodiment of the microparticles is, of course, represented by those microparticles that are obtainable or are produced by means of a process as has been defined above, either in general or in the form of any preferred embodiment of the said process.

Finally, the invention also relates to microparticles, compositions and methods as defined in any one of the remaining

As regards the determination of the immunological activity for the microparticles containing active substance, this must be carried out in a manner appropriate to each individual immunologically active substance. Where the determination is effected in the form of animal trials, a certain quantity of the immunologically active substance incorporated in the starch microparticles may be injected, if appropriate with at least one adjuvant, possibly after these microparticles have been previously enzymatically dissolved under mild conditions, and the immunological response is compared, after a suitable time interval after administration, with the response obtained after injection of a corresponding quantity of the same immunologically active substance in a suitable solution. Where the evaluation is made in vitro, for example in test tubes or in cell culture, the immunologically active substance is preferably made fully available before the evaluation by the starch microparticles being enzymatically dissolved under mild conditions, after which the antigenic activity is determined and compared with the activity for a control solution having the same concentration of the immunologically active substance in question. In any event, the evaluation shall include any non-specific effects of the degradation products of the starch microparticles.

The invention will now be explained further with reference to the following non-limiting examples. In these, as in the rest of the text, unless otherwise stated the percentages quoted relate to percentage by weight.

### EXAMPLES

### Example 1

### Preparation of OVA Starch Particles 40-100μ (Batch D-018)

Immobilization of OVA in starch microspheres produced from highly branched, sheared starch. All utensils were detoxified at 180° C. for 3 hours and autoclaved prior formulation.

A starch solution (30%) of highly branched, shared starch with an av. mol. wt. 408 kDa, a PEG solution (37.5% av. mol. wt. 20 kDa) and an OVA solution (0.38 mg/ml, purified from polymeric residues by means of gel chromatography) were prepared in 10 mM sodium phosphate buffer pH=7.4. The temperature of the starch solution was adjusted to 50° C. and the other solutions to approx. 23° C. The starch solution (14 g) was mixed with the OVA solution (4.69 ml) in an 250 ml IKA reactor equipped with an anchor propeller. The PEG solution (214 g) was added whilst stirring. The starch droplets were solidified at 20° C. for 7 h thereafter 37° C. for 17 hours. The starch microspheres containing OVA were washed with 10 mM sodium phosphate buffer pH=6.4, and freeze-dried.

The yield of dry particles in the range of 40-100μ was 70%.

The dried microspheres were dissolved by enzymatic action with a-amylase and amyloglucosidase for determining the protein and starch yield, and the protein loading. The loading of OVA was 0.40 μg/mg (analyzed by ELISA) giving a yield of 100%. The mean particle determined with a Malwern Mastersizer was 77 μm.

ELISA Analyzing OVA

Plates were coated with 50 μl/well with 20 μg/ml anti-OVA (Biodesign) in PBS at 4° C. over night. The wells were quenched with 100 μl 1% BSA for 1 h 37° C., Samples and standard were diluted PBS containing 0.2% BSA and subsequently diluted in a non-adsorbing plate 1+1 before transferring 50 μl to the ELISA plate and incubating for 1 h at 37° C. 50 μl anti-OVA-HRPO (RDI) was diluted 6000 times in 0.05% Tween 20 and applied into the wells and left 1 h at 37° C. OPD was used as substrate. Between all the steps the plates were washed with 0.1% Tween 20 in PBS.

### Example 2

### Preparation of OVA Starch Particles Containing Alum, 40-100μ (Batch D-004)

Immobilization of OVA in starch microspheres produced from highly branched, sheared starch. All utensils were and autoclaved prior formulation.

An OVA solution (1 mg/ml)was prepared in WFI of which 5.3 ml was mixed with 10.6 ml ALUM gel (Superfos Alhydrogel 2%) and left binding during 30 minutes.

A starch solution (30%) of highly branched, shared starch with an av. mol. wt. 529 kDa and a PEG solution (42% av. mol. wt. 20 kDa) were prepared in a 10 mM sodium phosphate buffer pH=6.4

The temperature of the starch solution was adjusted to 50° C. and the other solutions to approx. 38° C. The starch solution (12 g) was mixed with the OVA-ALUM solution (5.4 ml) and buffer (6.6 ml) in an 250 ml IKA reactor equipped with an anchor propeller. The PEG solution (175 g) was added whilst stirring. The starch droplets were solidified at 20° C. for 7 h thereafter 37° C. for 17 hours. The starch microspheres containing OVA were washed with 10 mM sodium phosphate buffer pH=6.4, and freeze-dried.

The yield of dry particles in the range of 40-100 μm was 58%.

The dried microspheres were dissolved by enzymatic action with a-amylase and amyloglucosidase for determining the protein and starch yield, and the protein loading. The theoretic load of OVA was 0.54 μg/mg. The mean particle determined with a Malwern Mastersizer was 70 μm.

### Example 3

### Preparation of OVA Starch Particles <10μ (Batch D-027)

Immobilization of OVA in starch microspheres produced from highly branched, sheared starch. All utensils were detoxified at 180° C. for 3 hours and autoclaved.

A starch solution (30%) of highly branched, shared starch with an av. mol. wt. 408 kDa, a PEG solution (37.5% av. mol. wt. 20 kDa) and an OVA solution (0.31 mg/ml, purified from polymeric residues by means of gel chromatography) were prepared in 10 mM sodium phosphate buffer pH=7.4. The temperature of the starch solution was adjusted to 50° C. and the other solutions to approx, 23° C. The starch solution (1.6 g) was mixed with the OVA solution (0.50 ml) in an 50 ml cerbo beaker. The PEG solution (20 ml) was added. Microdroplets were formed by an ultra turrax (IKA T-25). The starch droplets were solidified at 4° C. for approximately 24 h thereafter 37° C. for approximately 24 hours. The starch microspheres containing OVA were washed with 10 mM sodium phosphate buffer pH=6.4, and freeze-dried.

The dried microspheres were dissolved by enzymatic action with a-amylase and amyloglucosidase for determining the protein and starch yield, and the protein loading. The loading of OVA was 0.13 μg/mg as determined by ELISA giving a yield of 43%. The mean particle size determined using light microscopy was 3.3 μm.

### Example 4

### Preparation of OVA Starch Particles <10 μm by Soaking (Batch D-015, Kla01001)

Immobilization of OVA was done by soaking into preformed microspheres produced from highly branched, sheared starch. All utensils were detoxified at 180° C. for 3 hours and autoclaved.

A starch solution (30%) of highly branched, sheared starch with an av. mol. wt. 408 kDa and a PEG solution (37.5% at. mol. wt. 20 kDa) were prepared in 10 mM sodium phosphate buffer pH=7.4. The temperature of the starch solution was adjusted to 50° C. and the other solutions to approx. 23° C. The starch solution (1.6 g) was mixed with the OVA solution (0.50 ml) in an 50 ml cerbo beaker. The PEG solution (20 ml) was added. Microdroplets were formed by an ultra turrax (IKA T-25). The starch droplets were solidified at 4° C. for approximately 24 h thereafter 37° C. for approximately 24 hours. The placebo starch microspheres were washed with 10 mM sodium phosphate buffer pH=6.4, and freeze-dried.

An OVA solution (1.0 mg/ml, purified from polymeric residues by means of gel chromatography) was prepared in WFI. The OVA solution (300 μl) was mixed with WFI (500 μl) and subsequently 505 mg placebo starch microspheres. The mixture was incubated 37° C. for 30 minutes.

The dried microspheres were dissolved by enzymatic action with a-amylase and amyloglucosidase for determining the protein and starch yield, and the protein loading. The loading of OVA was 0.23 μg/mg as determined by ELISA. The mean particle size as determined with light microscopy was 3.2 μm.

### Example 5

### Procedure for Coating of Starch Microspheres Containing OVA. (Coat 345, RG 502H)

The OVA-containing starch microspheres obtained in Examples 2 and 3 were coated with a release-controlling shell made from PLGA by means of air suspension technology according to WO97/14408 with the RG502H (Boehringer Ingelheim). After the coating operation, the coating was dissolved with a mixture of methylene chloride and acetone in a ratio of 1:3 and, after these solvents have been washed away, for example by repeated centrifugation, the microspheres were dissolved with α-amylase. The OVA content was determined by analysis with ELISA. The protein content was around 0.02 percent by weight. The, release kinetics for OVA from the coated microspheres was determined in vitro. With this process, parentally administrable microspheres can thus be produced so as to be suitable for vaccine delivery,

The microparticles thus obtained were then subjected to an experiment concerning release in vitro in 30 mM sodium phosphate, pH 7.4, containing 82 mM sodium chloride, 3 mM sodium azide, 0.5 mM calcium chloride, 0.2% bovine serum albumin and 185 U/l α-amylase, at 37° C. with intermittent agitation. The studies were performed by suspending 40 mg of microspheres in 1.5 mL of buffer. At specific times 1 mL aliquots of said buffer were removed and replaced by fresh buffer.

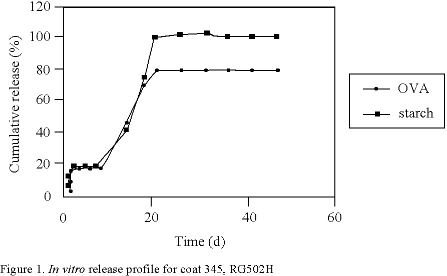

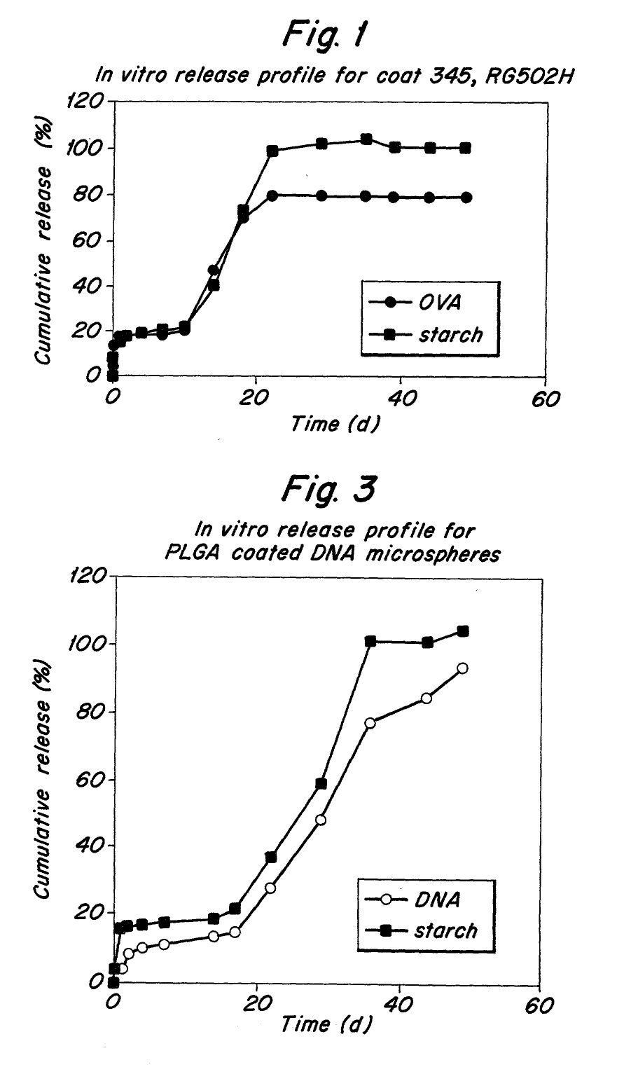

FIG. 1. In vitro release profile for coat 345, RG502H

### Example 6

### In vivo Assessment of Immune-activity

Experiment 1

Female BalbC mice 20-22 g (6-8 weeks old) were used in the study, 8 animals/group. The animals were primed s.c. on day 0 with 1 μg OVA 1 s.c. In the neck and subsequently boostered s.c. the neck at day 21 with 1 μg OVA. A CMC solution was used as diluent. Blood samples were taken on day 0, 21, 35, 49, 63, 77 and 91 from the tail vein. The blood samples were stored at 4° C. over night and centrifuged at 3000 rpm for 10 minutes. From the sera of each animal 5 μl sera were transferred to a pooled group serum. Before ELISA analysis the samples were stored at −20° C.,

ELISA Analysis of Total IgG

Plates were coated with 50 μl/well with 5 μg/ml OVA (Sigma A-5503) in PBS at 4° C. over night. The wells were quenched with 100 μl 1% BSA for 1 h 37° C. Monoclonal IgG (Sigma A-6075 was used as a standard. Sera and standard were diluted PBS containing 0.2% BSA and subsequently diluted in a non-adsorbing plate 1+1 before transferring 50 μl to the ELISA plate and incubating for 1 h at 37° C. 50 μl anti-mouse-IgG-HRPO was diluted 6000 times in 0.05% Tween 20 and applied into the wells and left 1 h at 37° C. OPD was used as substrate. Between all the steps the plates were washed with 0.1% Tween 20 in PBS.

TABLE 3
Overview of immunisations, Experiment 1

| Group | Prima Day 0: 1 μg OVA | Booster Day 21: 1 μg OVA |
|---|---|---|
| 1 | ALUM | ALUM |
| 2 | Coated starch microspheres | Coated starch microspheres |
| | PLGA resomer RG 502H | PLGA resomer RG 502H |
| 3 | Starch microspheres 40-100 μ | Starch microspheres 40-100 μ |
| 4 | Starch microspheres <10 μ | Starch microspheres <10 μ |
| 5 | Soaked starch microspheres | Soaked starch microspheres |
| | <10 μ | <10 μ |

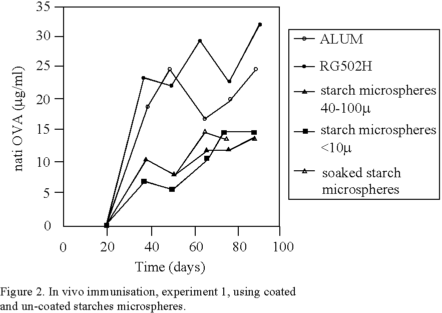

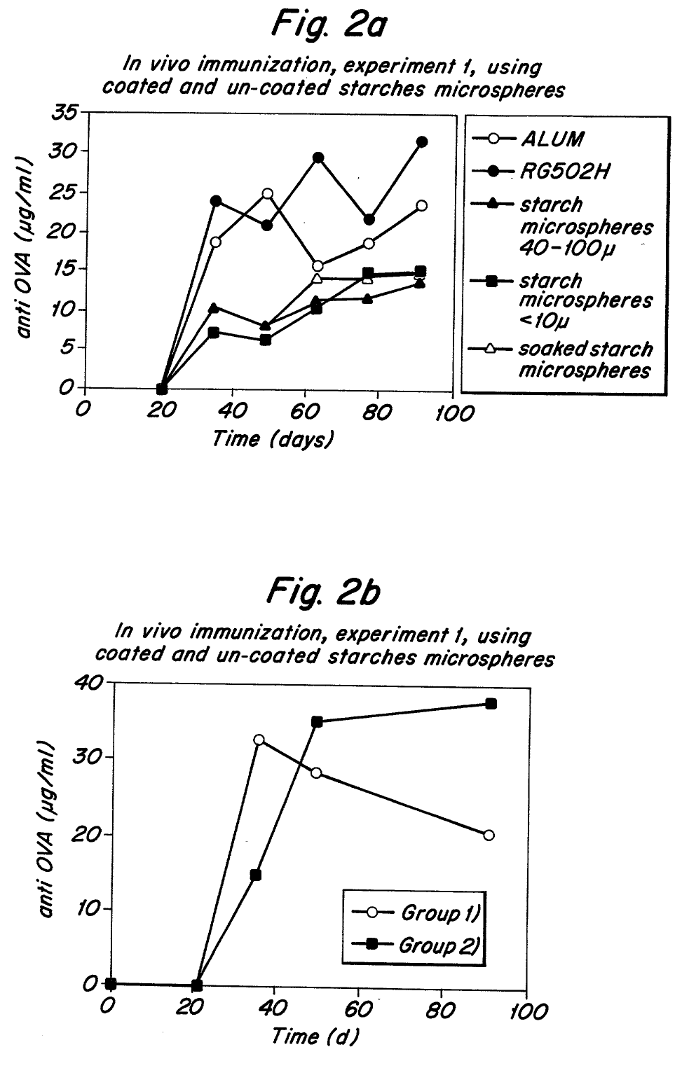

FIG. 2. In vivo immunisation, experiment 1, using coated and un-coated starches microspheres.

Experiment 2

Female BalbC mice 20-22 g (6-8weeks old) were used in the study, 8 animals/group. The animals were primed s.c. on day 0 with 2.5 μg OVA a.c. In the neck and subsequently boostered s.c. the neck at day 21 with 2.5 μg OVA. A starch solution was used as diluent. Blood samples were taken on day 0, 21, 35, 49 and 91 from the retroorbital point. From the sera of each animal 5 μl sera were transferred to a pooled group sera, Before ELISA analysis the samples were stored at −20° C.

TABLE 4
Overview of immunisations, Experiment 1

| Group | Prime Day 0: 1 μg OVA | Booster Day 21: 1 μg OVA |
|---|---|---|
| 1 | ALUM | ALUM |
| 2 | ALUM | Coated ALUM containing |
| | | starch microspheres |
| | | PLGA resomer RG 502H |

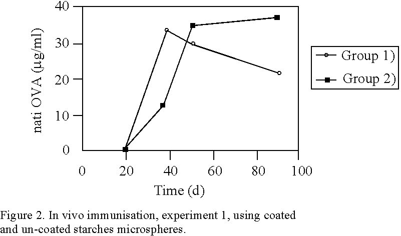

FIG. 2. In vivo immunisation, experiment 1, using coated and un-coated starches microspheres.

### Example 7

### Preparation of DNA Plasmid Starch Particles 40-100μ

Immobilization of plasmid DNA (5 kb, pDNA-3, INVITROGEN) in starch microspheres produced from highly branched, sheared starch. All utensils were autoclaved prior formulation.

A starch solution (31.5%) of highly branched, sheared starch with an av. mol. wt. 408 kDa prepared in distilled water, a PEG solution (40% av. mol. wt. 20 kDa) and a DNA-plasmid solution (1.4 mg/ml) were prepared in 10 mM sodium phosphate buffer with 1 mM EDTA pH=7.0. The temperature of the starch solution was adjusted to 50° C. and the other solutions to 37° C. The starch solution (1.4 g) was mixed with 0.446 ml 10 mM sodium phosphate buffer containing 1 mM EDTA pH=7.0 and DNA solution (0.154 ml) in an 50 ml beaker equipped with an anchor propeller. The PEG solution (22 g) was added whilst stirring. The starch droplets were solidified at 4° C. for 4 h thereafter 37° C. for 17 hours. The starch microspheres containing DNA were washed with 1 mM EDTA solution and subsequently with an ethanol solution, and dried in an LAUF-hood.

The yield of dry particles in the range of 40-100μ was 60%.

The dried microspheres were dissolved by enzymatic action with α-amylase for determining the DNA and starch yield, and the protein loading. The loading of DNA was 0.19 μg/mg (fluorometically analyzed by Pico Green method, Molecular Probes) giving a yield of 80%, the starch yield was 95%.

The DNA-containing starch microspheres obtained in Example 7 were coated with a release-controlling shell made from PLGA by means of air suspension technology according to WO97/14408 with the a polymer composition consisting of 75% RG502H and 25% RG 756 (Boehringer Ingelheim). After the coating operation, the DNA load was assessed by dissolving the coating with a mixture of methylene chloride and acetone in a ratio of 1:3 and, after these solvents had been washed away, for example by repeated centrifugation, the microspheres were dissolved with α-amylase. The DNA content was determined, by analysis with Pico Green Metod, Molecular Probes. The release kinetics for DNA from the coated microspheres were determined in vitro. With this process, parentally administrable microspheres can thus be produced so as to be suitable for vaccine delivery.

The microparticles thus obtained were then subjected to an experiment concerning release in vitro in 30 mM sodium phosphate, pH 7.4, at 37° C. with intermittent agitation. The studies were performed by suspending 40 mg of microspheres in 1.5 mL of buffer. At specific times 1 mL aliquots of said buffer were removed and replaced by fresh buffer.

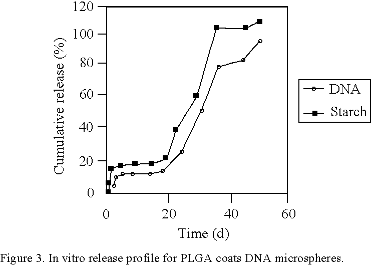

FIG. 3. In vitro release profile for PLGA coated DNA microspheres.

1. A vaccine composition which comprises an immunologically active substance embedded in microparticles essentially consisting of starch having an amylopectin content exceeding 85% by weight, of which at least 80% by weight has an average molecular weight within the range of 10-10000 kDa.

2. A vaccine composition according to claim 1, in which said starch has an amino acid nitrogen content of less than 50 μg per gram dry weight of starch, and in which there are no covalent chemical cross-linking between the starch molecules.

3. A composition according to claim 1, in which the starch has a purity of at most 20 μg, preferably at most 10 μg, and more preferably at most 5 μg, amino acid nitrogen per g dry weight of starch.

4. A composition according to claim 1, in which the starch has an amylopectin content with said molecular weight exceeding 95% by weight, preferably exceeding 98% by weight.

5. A composition according to claim 1, in which the molecular weight of said amylopectin has been reduced such that at least 80% by weight of the material is within the range of 100-4000 kDa, preferably 200-1000 kDa, and more preferably 300-600 kDa.

6. A composition according to claim 1, in which the starch is such that it can be dissolved in a concentration exceeding 25% by weight in water.

7. A composition according to claim 1, in which the starch essentially lacks covalently bonded extra chemical groups of the type which are sound in hydroxyethyl starch.

8. A composition according to claim 1, in which the starch has an endotoxin content of less than 25 EU/g and contains less than 100 microorganisms per gram.

9. A composition according to claim 1, in which the starch has been essentially purified from surface-localized proteins, lipids and endotoxins by means of washing with aqueous alkali solution and purified from internal proteins by means of ion exchange chromatography, preferably anion exchange chromatography.

10. A composition according to claim 1, in which said starch also contains 2-15% by weight of amylose, having an average molecular weight within the range of 2.5-70 kDa, preferably 5-45 kDa, in which the percentage share by weight is calculated on the basis of dry weight of starch.

11. A composition according to claim 1, in which the immunoactivity of said immunologically active substance is at least 80%, preferably at least 90% and more preferably essentially maintained compared with the immunoactivity exhibited by said substance prior to its incorporation in the starch.

12. A composition according to claim 1, wherein said microparticles are biodegradable in vitro in the presence of alpha-amylase and/or amyloglucosidase.

13. A composition according to claim 1, wherein said microparticles are biodegradable and are eliminated from tissue after subcutaneous or intramuscular administration.

14. A composition according to claim 1, wherein said microparticles have a release-controlling shell of at least one film-forming biocompatible and biodegradable polymer.

15. A composition according to claim 14, in which the polymer is a homopolymer or copolymer containing alpha-hydroxy acid units.

16. A composition according to claim 15, in which the alpha-hydroxy acid is lactic acid and/or glycolic acid.

17. A vaccine composition according to claim 1, wherein the immunologically active substance is derived from at least one of the following classes of vaccines: NMDA glutamate receptor vaccines, whole cell vaccines, tumour-antigen vaccines, peptide vaccines, allergoid vaccines, anti-iodiotype vaccines, dendritic cell-based vaccines, subunit and recombinant subunit vaccines, DNA vaccines, live viral vector vaccines, live bacterial vaccines and self antigen vaccines.

18. A vaccine composition according to claim 17, which also contains an adjuvant.

19. A vaccine composition according to claim 18, where said adjuvant is derived from: aluminium salts, saponins, lipid-A or derivatives thereof, immunostimulatory oligonucleotides or cytokines.

20. A process for producing a vaccine composition comprising an immunologically active substance embedded in microparticles which process comprises:
a) preparing an aqueous starch solution, comprising starch which has an amylopectin content exceeding 85% by weight, and in which the molecular weight of the said amylopectin has been reduced such that at least 80% by weight of the material is within the range of 10-10000 kDa, the starch concentration of the solution being at least 20% by weight,
b) combining the immunologically active substance with the starch solution under such conditions that a composition in the form of a solution, emulsion or suspension of said agent in the starch solution is formed,
c) mixing the composition obtained in step b) with an aqueous solution of a polymer having the ability of forming a two-phase aqueous system, thereby forming an emulsion of starch droplets which contain the immunologically active substance as an inner phase in an outer phase of said polymer solution,
d) causing or allowing the starch droplets obtained in step c) to gel into starch particles through the natural capacity of the starch to solidify,
e) drying the starch particles, preferably after prior removal of said outer phase through washing, and
f) optionally applying a release-controlling shell of a biocompatible and biodegradable polymer, preferably by air suspension technology, to the dried starch particles.

21. A process for producing a vaccine composition comprising an immunologically active substance chosen from at least one of the following classes of vaccines: NMDA glutamate receptor vaccines, whole cell vaccines, tumour-antigen vaccines, peptide vaccines, allergoid vaccines, anti-iodiotype vaccines, dendritic cell-based vaccines, subunit and recombinant subunit vaccines, DNA vaccines, live viral vector vaccines, live bacterial vaccines, and self antigen vaccines; comprising
a) preparing an aqueous solution of the immunologically active substance,
b) combining the immunologically active substance with starch microspheres under such conditions that the substance becomes associated with the microspheres,
c) exposing the resulting mixture to evaporation or other drying.

22. A process according to claim 20 or 21, in which the starch is as defined in claim 2.

23. A process according to claim 20, in which in step a) a solution is prepared having a starch concentration of at least 30% by weight.

24. A process according to claim 20, in which in step a) a solution is prepared having a starch concentration of at most 50% by weight, preferably at most 45% by weight.

25. A process according to claim 20, in which the aqueous starch solution in step a) is prepared with accompanying autoclaving of the same.

26. A process according to claim 20, in which in step b) the immunologically active substance is combined with the starch solution at a temperature of at most 60° C., preferably 20-45° C., especially 30-37° C.

27. A process according to claim 20, in which in step b) a composition is formed in which the weight ratio between starch and immunologically active substance is within the range of 3:1 to 10000:1, preferably 3:1 to 100:1.

28. A process according to claim 20, in which in step c) the polymer is used in a concentration in said aqueous solution of at least 20% by weight, preferably at least 30% by weight.

29. A process according to claim 20, in which in step c) the polymer is used in a concentration in said aqueous solution of at most 45% by weight, preferably 30-40% by weight.

30. A process according to claim 20, in which the mixing in step c) is performed at a temperature within the range of 4-50° C., preferably 10-40° C., especially 10-37° C.

31. A process according to claim 20, in which the mixing in step c) is performed with the aid of at least one static mixer.

32. A process according to claim 20, in which in step c) the polymer solution is added to the composition in at least two steps, in which at least one of the additions is effected after the emulsion has begun to be created.

33. A process according to claim 20, in which in step c) polyethylene glycol is used as the aqueous polymer.

34. A process according to claim 33, in which the polyethylene glycol has an average molecular weight of 5-35 kDa, preferably 15-25 kDa, especially about 20 kDa.

35. A process according to claim 20, in which the solidification in step d) is performed at at least two temperatures, in which the initiation is effected at a lower temperature than the termination.

36. A process according to claim 35, in which the solidification is initiated within the range of 1-20° C., preferably 1-10° C., especially around 4° C., and is terminated within the range of 20-55° C., preferably 25-40° C., especially around 37° C.

37. A process according to claim 20, in which the drying in step e) is performed in the form of spray-drying, freeze-drying or vacuum-drying, preferably freeze-drying.

38. A process according to claim 20, in which, as said immunologically active substance, a substance is incorporated which gives any one of the following classes of vaccines; NMDA glutamate receptor vaccines, whole cell vaccines, tumour-antigen vaccines, peptide vaccines, allergoid vaccines, anti-iodiotype vaccines, dendritic cell-based vaccines, subunit and recombinant subunit vaccines, DNA vaccines, live viral vector vaccines, live bacterial vaccines and self antigen vaccines.

39. A process according to claim 20, in which in step c) starch droplets are formed which give the size required for the microparticles, preferably a mean particle diameter, in the dry state, within the range of 10-200 μm, preferably 20-100 μm, more preferably 20-80 μm.

40. A process according to claim 20, in which after step d) the microparticles are washed, through filtration, and optionally sieved in order to obtain the desired particle size distribution.

41. Use of microparticles as defined in claim 1, for the manufacture of a vaccine.

42. A method of immunizing a mammal, especially a human being, in need thereof, which comprises parenteral administration, preferably via injection, of a vaccine composition as defined in claim 1 to said mammal.

43. A method of inducing an immune response in a mammal, especially a human being, comprising administering a microparticle having the characteristics as defined in claim 1.

44. A microparticle having a starch matrix, which microparticle contains an immunologically active substance, the majority of the immunologically active substance being associated in the starch matrix.

45. A microparticle having a starch matrix, which microparticle contains an immunologically active substance, the majority of the immunologically active substance being associated in the starch matrix, and which is coated with an organic polymer with selective solubilisation properties.

46. A microparticle according to claim 44 or 45, which starch has an amylopectin content exceeding 85% by weight, in which the molecular weight of the said amylopectin has been reduced such that at least 80% by weight of the material is within the range of 10-10000 kDa, and which has an amino acid nitrogen content of less than 50 μg per g dry weight of starch.

47. A vaccine composition comprising:
a) one or more immunologically active substances selected from the groups: viral antigens, bacterial antigens, parasitic antigens, recombinant proteins, whole cell antigens, live attenuated virus, live attenuated bacteria, tumour antigens and self antigens, and
b) microparticles of starch having an amylopectin content exceeding 85% by weight, in which the molecular weight of the said amylopectin has been reduced such that at least 80% by weight of the material is within the range of 10-10000 kDa, and which has an amino acid nitrogen content of less than 50 μg per g dry weight of starch, the bulk of the immunologically active substance being associated with the starch particle.

48. A vaccine composition comprising: p1 a) one or more immunologically active substances selected from the groups: viral antigens, bacterial antigens, parasitic antigens, recombinant proteins, whole cell antigens, live attenuated virus, live attenuated bacteria, tumour antigens and self antigens, and
b) an immunomodulatory molecule selected from: lipid A or analogues thereof, saponins, immunostimulatory oligonucleotides, mineral salts and low molecular weight thiols, for example cysteamine, and
c) a starch microparticle.

49. Parenterally administrable microparticles containing associated immunologically active substances selected from the groups: viral antigens, bacterial antigens, parasitic antigens, recombinant proteins, whole cell antigens, live attenuated virus, live attenuated bacteria, tumour antigens and self antigens, the particles being made from starch, and upon suspension in an aqueous medium at physiological conditions over 50% of the associated immunologically active substance being released within 3 hours.

50. Method for entrapping an immunologically active substance comprising adding a solution of immunologically active substance selected from the groups: viral antigens, bacterial antigens, parasitic antigens, recombinant proteins, whole cell antigens, live attenuated virus, live attenuated bacteria, tumour antigens and self antigens to a drug preparation of starch particles followed by exposing the resulting mixture to evaporation.

51. Method of inducing an immune response in a mammal, especially a human being, comprising administering by intramuscular, subcutaneous, intradermal or nasal route a composition comprising an immunologically active substance selected from the groups: viral antigens, bacterial antigens, parasitic antigens, recombinant proteins, whole cell antigens, live attenuated virus, live attenuated bacteria, tumour antigens and self antigens and starch microparticles.

52. A method for inducing an immune response for oral administration comprising administering by oral route a composition comprising immunologically active substance selected from the groups: viral antigens, bacterial antigens, parasitic antigens, recombinant proteins, whole cell antigens, live attenuated virus, live attenuated bacteria, tumour antigens and self antigens entrapped within starch microparticles having one or more coatings of enteric polymer(s).

53. A composition according to claim 48, where the starch is as defined in claim 1.
